(19) United States
(12) Patent Application Publication
(10) Pub. No.: US 2026/0174323 A1
(43) Pub. Date: Jun. 25, 2026

(54) QUALITY CONTROL METHOD AND QUALITY CONTROL SYSTEM FOR DATA ANNOTATION ON FUNDUS IMAGE

(71) Applicant: SHENZHEN SIBRIGHT TECHNOLOGY CO., LTD., Shenzhen (CN)

(72) Inventors: Juan Wang, Shenzhen (CN); Juan Hu, Shenzhen (CN); Zhigang Hu, Shenzhen (CN); Ming Lai, Shenzhen (CN)

(73) Assignee: SHENZHEN SIBRIGHT TECHNOLOGY CO., LTD., Shenzhen (CN)

(21) Appl. No.: 19/538,102

(22) Filed: Feb. 12, 2026

Related U.S. Application Data
(63) Continuation-in-part of application No. 18/259,390.

(30) Foreign Application Priority Data
Dec. 28, 2020 (CN) 202011588182.0

(51) Int. Cl. A61B 3/00; A61B 3/12; A61B 3/14

(52) U.S. Cl. CPC A61B 3/0025; A61B 3/12; A61B 3/1225; A61B 3/14; G06T 2207/20081

(57) ABSTRACT

Some embodiments of the disclosure provide a quality control method for data annotation on a fundus image. In some examples, the method includes: acquiring a plurality of fundus images; performing standardization processing on the fundus images to obtain a plurality of standardized fundus images; performing preliminary filtering on quality of the standardized fundus images to acquire a plurality of qualified fundus images; preparing a target fundus image set; a plurality of first annotation doctors respectively annotating the images of the target fundus image set, to acquire a plurality of groups of doctor annotation results; calculating, on the basis of the doctor annotation results, self-consistency and gold-standard consistency of the corresponding first annotation doctors, to acquire the doctor annotation results of the first annotation doctors satisfying a preset condition as target annotation results; and gathering a plurality of groups of target annotation results to acquire a final annotation result.

### CROSS REFERENCE TO RELATED APPLICATIONS

This application is a Continuation in Part of U.S. patent application Ser. No. 18/259,390, filed on Jun. 26, 2023, which is the United State national stage entry under 37 U.S.C. 371 of PCT/CN2021/091225, filed on Apr. 29, 2021, which claims priority to Chinese patent application number 202011588182.0, filed on Dec. 28, 2020, the disclosure of which are incorporated by reference herein in their entireties.

### FIELD OF THE DISCLOSURE

The disclosure relates generally to the field of machine learning in medical systems and methods. More specifically, the disclosure relates to computerized quality control methods and quality control systems for data annotation on fundus images.

### BACKGROUND

With the development of artificial intelligence technology, supervised learning technology based on machine learning has been applied in more and more fields. Especially in the field of medical imaging, supervised learning technology based on machine learning is a big success. In supervised learning, a machine learning model is trained using a training set consisting of training data (e.g., fundus images) and annotation results of the training data (e.g., diabetic retinopathy staging), so the data annotation quality of the training data is crucial to the training of the model.

Currently, in order to make the annotation result of training data to be more accurate, professional annotators such as professional ophthalmologists are often allowed to annotate training data and perform quality control on the annotation result in combination with quality control methods. For example, literature (CN110991486 A) discloses a method for multi-person collaborative image annotation quality control, in which gold-standard data is input into an annotation package according to a pre-set proportion to verify the annotation quality of any annotation package annotated by an annotation user. In a multi-person fitting step, an image is distributed to a plurality of users, annotation results of the image by the plurality of users are collected, and a real label is obtained after repeated labels are obtained. However, the accuracy of the annotation results of the training data needs to be improved.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, a first aspect of the present disclosure provides a quality control method for data annotation on a fundus image, including: acquiring a plurality of fundus images; performing standardization processing on each of the plurality of fundus images to obtain a plurality of standardized fundus images; performing preliminary filtering on quality of each of the plurality of standardized fundus images to obtain a plurality of qualified fundus images; preparing a target fundus image set, the target fundus image set includes a data set to be calibrated including the plurality of qualified fundus images, a gold-standard data set including a first preset number of gold-standard fundus images with a known correct annotation result, and a self-consistency determination data set composed of at least one image in the data set to be calibrated, and taking each image of the target fundus image set as a respective target fundus image; annotating respective images of the target fundus image set by a plurality of first annotation doctors respectively to obtain a plurality of groups of doctor annotation results, the doctor annotation results include at least one determination result, the determination result at least includes disease information of no obvious abnormality or of a disease; calculating self-consistency and gold-standard consistency of the corresponding first annotation doctors based on the doctor annotation results to acquire the doctor annotation results of the first annotation doctors satisfying a preset condition as target annotation results, obtaining the self-consistency by taking any one of two groups of annotation results of the doctor annotation result of each image in the self-consistency determination data set and the doctor annotation result of an image, which is repeated with respective image in the self-consistency determination data set, in the data set to be calibrated as a first group of annotation results and taking the other group as a second group of annotation results and performing evaluation using a self-consistency determination and evaluation method, acquiring the gold-standard consistency by taking the correct annotation result of the gold-standard data set as a first group of annotation results and the doctor annotation result of each image in the gold-standard data set as a second group of annotation results and using a gold-standard consistency determination and evaluation method; gathering a plurality of sets of the target annotation results to obtain a final annotation result. In this case, based on the gold-standard data set and the self-consistency determination data set, the doctor annotation result of the first annotation doctor that satisfies the preset conditions may be obtained as the target annotation result and gathered. Thus, the accuracy of data annotation of the fundus image may be improved.

In addition, in the quality control method according to the first aspect of the present disclosure, optionally, the preset condition is that the self-consistency is greater than a self-consistency threshold value and the gold-standard consistency is greater than a gold-standard consistency threshold value. Thus, the preset condition may be determined based on the self-consistency threshold value and the gold-standard consistency threshold value.

In addition, in the quality control method according to the first aspect of the present disclosure, optionally, when the doctor annotation result of the first annotation doctor does not meet the preset condition, each image in the target fundus image set is re-annotated by the second annotation doctor until the doctor annotation result meeting the preset condition is obtained as the target annotation result. Thus, a target annotation result may be obtained.

In addition, in the quality control method according to the first aspect of the present disclosure, optionally, the self-consistency determination method is to calculate a disease self-consistency of each of the disease determined by each first annotation doctor using a quadratic weighted kappa coefficient and to weight each disease self-consistency to calculate the self-consistency of each first annotation doctor; the gold-standard consistency determination method is to calculate the gold-standard consistency of each of the disease determined by each first annotation doctor using a quadratic weighted kappa coefficient and to weight the gold-standard consistency of the disease to calculate the gold-standard consistency of each first annotation doctor. Thus, the self-consistency of each first annotation doctor may be calculated based on the self-consistency determination method and the gold-standard consistency of each first annotation doctor may be calculated based on the gold-standard consistency determination method.

In addition, in the quality control method according to the first aspect of the present disclosure, optionally, the quadratic weighted kappa coefficient κ is

$$κ = 1 - \frac{{\sum }_{i , j} {W}_{ij} {X}_{ij}}{{\sum }_{i , j} {W}_{ij} {E}_{ij}} .$$

Here, Wᵢⱼ represents a quadratic weighting coefficient, Xᵢⱼ represents a number of the target fundus images for which the determination result in the first group of annotation results is i and the determination result in the second group of annotation results is j, and Eᵢⱼ represents an expected number of the target fundus images for which the determination result in the first group of annotation results is i and the determination result in the second group of annotation results is j. Thus, it is able to inspect consistency between the first group of annotation results and the second group of annotation results.

In addition, in the quality control method according to the first aspect of the present disclosure, optionally, the self-consistency threshold value and the gold-standard consistency threshold value are determined through analyzing target self-consistency and target gold-standard consistency of doctors with different threshold value annotation using abnormality detection. Thus, the self-consistency threshold value and the gold-standard consistency threshold value may be determined.

In addition, in the quality control method according to the first aspect of the present disclosure, optionally, the abnormality detection is to acquire the target self-consistency of the doctors with different threshold value annotation and calculate a self-consistency mean value μ₀ and a self-consistency variance σ₀, under the assumption that the target self-consistency satisfies a Gaussian distribution, the self-consistency threshold value is μ₀−1.96×σ₀, and to acquire the target gold-standard consistency of the doctors with different threshold value annotation and calculate a gold-standard consistency mean value μ₁ and a gold-standard consistency variance σ₁, under the assumption that the target gold-standard consistency satisfies a Gaussian distribution, the gold-standard consistency threshold value is μ₁−1.96×σ₁. Thus, the self-consistency threshold value and the gold-standard consistency threshold value may be determined.

In addition, in the quality control method according to the first aspect of the present disclosure, optionally, the gathering is to compare each annotation result of each target fundus image in a plurality of groups of the target annotation results using an absolute majority voting method to determine the final annotation result of each target fundus image, and if the final annotation result is not able to be determined, the target fundus image is annotated as a difficult fundus image, and the difficult fundus image is annotated and arbitrated to obtain the final annotation result. Thus, the final annotation result may be obtained based on the absolute majority voting method.

In addition, in the quality control method according to the first aspect of the present disclosure, optionally, the gathering is to compare each annotation result of each target fundus image in a plurality of groups of the target annotation results, and in the case that each annotation result is consistent, taking the annotation result as the final annotation result of the target fundus image, while in the case that a plurality of annotation results are inconsistent, if the plurality of annotation results simultaneously include a same determination result and only one annotation result includes a determination result which is not identified in the other annotation results, the target fundus image is annotated as a fundus image to be quality-controlled, otherwise, the target fundus image is annotated as a difficult fundus image; quality control is performed on the fundus image to be quality-controlled and the final annotation result is obtained, and the difficult fundus image is annotated and arbitrated to obtain the final annotation result. In this case, the target fundus image may be divided into a target fundus image having a final annotation result, a fundus image to be quality-controlled, and a difficult fundus image and the final annotation result may be obtained by comparing each annotation result of each target fundus image among a plurality of groups of target annotation results.

In addition, in the quality control method according to the first aspect of the present disclosure, optionally, quality control is performed on the fundus image to be quality-controlled, and if it is determined that the unidentified determination result does not exist, the same determination result is taken as the final annotation result, while if it is determined that the unidentified determination result exists, the fundus image to be quality-controlled is taken as a difficult fundus image and the difficult fundus image is annotated and arbitrated to obtain the final annotation result. Thus, the fundus image to be quality-controlled may be divided into the fundus image to be quality-controlled having the final annotation result and the difficult fundus image, and the final annotation result may be obtained.

In addition, in the quality control method according to the first aspect of the present disclosure, optionally, the difficult fundus image is annotated and arbitrated by an arbitration doctor to obtain the final annotation result. Thus, the final annotation result of the difficult fundus image may be obtained.

In addition, in the quality control method according to the first aspect of the present disclosure, optionally, the standardization processing includes at least one of dividing the fundus images per the patient, unifying a name format of the fundus images, filtering out a non-fundus image, unifying a picture format of the fundus images, and unifying a background of the fundus images. Thus, the standardization processing on fundus images may be accomplished.

In addition, in the quality control method according to the first aspect of the present disclosure, optionally, the preliminary filtering includes dividing the standardized fundus images into at least two image quality grades including qualified and unqualified, the qualified fundus image is the standardized fundus image whose image quality grade is qualified. Thereby, the quality of the standardized fundus images may be preliminarily filtered to quickly obtain qualified fundus images.

In addition, in the quality control method according to the first aspect of the present disclosure, optionally, in the annotation, image quality of each image in the target fundus image set is classified into five image quality grades including very good, good, average, poor and very poor. In this case, the final annotation result may be subsequently determined in connection with a more detailed image quality grade.

In addition, in the quality control method according to the first aspect of the present disclosure, optionally, the disease includes at least one of diabetic retinopathy, hypertensive retinopathy, glaucoma, retinal vein occlusion, retinal artery occlusion, age-related macular degeneration, high myopia macular degeneration, retinal detachment, optic nerve disease, and congenital abnormalities of disc development. Thereby, at least one disease may be annotated.

In addition, in the quality control method according to the first aspect of the present disclosure, optionally, the preset condition is d_{self}≤D and d_{gold}≤D. Here, d_{self} is a self-evaluation index based on the self-consistency, d_{gold} is a gold-standard evaluation index based on the gold-standard consistency, and D is an evaluation index threshold value, the self-evaluation index d_{self} satisfies the formula: d_{self}=|J_{self}−κ_{self}|/κ_{self}×100%. Here, J_{self}=SE_{self}+SP_{self}−1, SE_{self} is sensitivity of the first annotation doctor obtained based on the two groups of annotation results for evaluating the self-consistency and SP_{self} is specificity of the first annotation doctor obtained based on the two groups of annotation results for evaluating the self-consistency, κ_{self} is the self-consistency of the first annotation doctor, the gold-standard evaluation index d_{gold} satisfies the formula: d_{gold}=|J_{gold}−κ_{gold}|/κ_{gold}×100%. Here, J_{gold}=SE_{gold}+SP_{gold}−1, SE_{gold} is, sensitivity of the first annotation doctor obtained based on the two groups of annotation results for evaluating the gold-standard consistency, and SP gold is the specificity of the first annotation doctor obtained based on the two groups of annotation results for evaluating the gold-standard consistency, and κ_{gold} is the gold-standard consistency of the first annotation doctor. Thus, the preset condition may be determined based on the evaluation index threshold value.

A second aspect of the present disclosure provides a quality control system for data annotation on a fundus image, including: an acquisition module, used for acquiring a plurality of fundus images; a standardization processing module, used for performing standardization processing on each of the fundus images to obtain a plurality of standardized fundus images; a preliminary filtering module, used for performing preliminary filtering on the quality of each of the standardized fundus images to obtain a plurality of qualified fundus images; a data preparation module, used for preparing a target fundus image set, the target fundus image set includes a data set to be calibrated including the plurality of qualified fundus images, a gold-standard data set including a first preset number of gold-standard fundus images with a known correct annotation result, and a self-consistency determination data set composed of at least one image in the data set to be calibrated, each image of the target fundus image set is taken as each target fundus image; a annotation module, used for acquiring a plurality of groups of doctor annotation results by a plurality of first annotation doctors respectively annotating each image in the target fundus image set, the doctor annotation results include at least one determination result, the determination result at least includes disease information of no obvious abnormality or of a disease; an evaluation module, used for calculating a self-consistency and a gold-standard consistency of a corresponding first annotation doctor based on the doctor annotation result to obtain the doctor annotation result of the first annotation doctor satisfying a preset condition as a target annotation result, the self-consistency is obtained by taking any one of two groups of annotation results of the doctor annotation result of each image in the self-consistency determination data set and the doctor annotation result of an image, which is repeated with respective image in the self-consistency determination data set, in the data set to be calibrated as a first group of annotation results and the other group as a second group of annotation results and performing evaluation using a self-consistency determination and evaluation method, the gold-standard consistency is obtained by taking the correct annotation result of the gold-standard data set as a first group of annotation results and the doctor annotation result of each image in the gold-standard data set as a second group of annotation results and using a gold-standard consistency determination and evaluation method; a gathering module, used for gathering the plurality of groups of the target annotation results to obtain a final annotation result. In this case, based on the gold-standard data set and the self-consistency determination data set, the doctor annotation result of the first annotation doctor that satisfies the preset conditions may be obtained as the target annotation result and gathered. Thus, the accuracy of data annotation of the fundus image may be improved.

According to the present disclosure, it is able to provide a quality control method and a quality control system for data annotation on fundus images with high accuracy.

### BRIEF DESCRIPTION OF THE DRAWINGS

Illustrative embodiments of the present disclosure are described in detail below with reference to the attached drawing figures.

FIG. 1 is a use scenario diagram illustrating a computerized quality control method for data annotation on a fundus image according to an embodiment of the disclosure.

FIG. 2 is a flowchart illustrating a computerized quality control method for data annotation on a fundus image according to an embodiment of the disclosure.

FIG. 3 is a block diagram illustrating a target fundus image set according to an embodiment of the disclosure.

FIG. 4 is a flowchart illustrating the computerized determination of a self-consistency threshold value according to an embodiment of the disclosure.

FIG. 5 is a statistical chart illustrating target self-consistency and target gold-standard consistency according to an embodiment of the disclosure.

FIG. 6 is a flowchart illustrating the manner in which the examples of the present disclosure are gathered.

FIG. 7 is a flowchart illustrating computerized quality control of a fundus image to be quality-controlled and obtaining a final annotation result according to an embodiment of the disclosure.

FIG. 8 is a block diagram showing a computerized quality control system for data annotation on a fundus image according to an embodiment of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

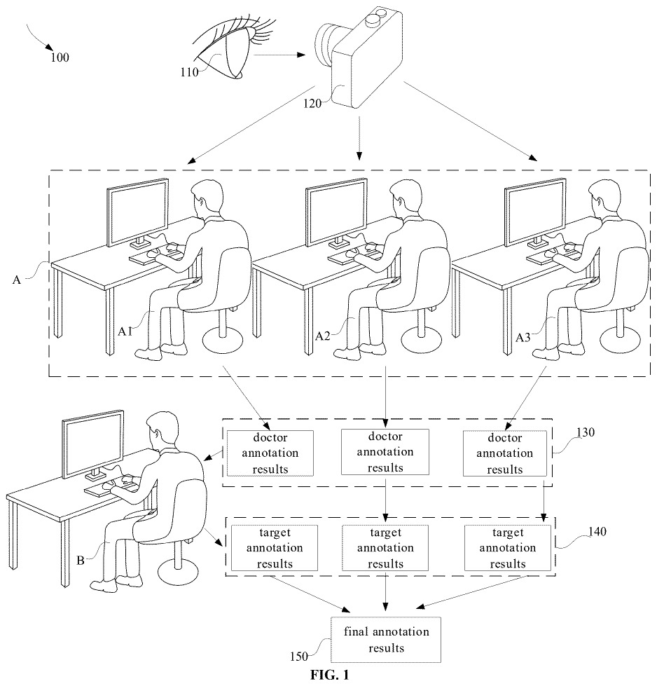

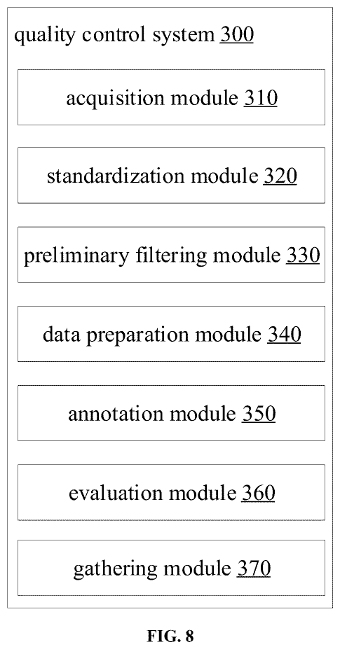

FIG. 1 is a use scenario diagram illustrating a use scenario 100 for a computerized quality control method for data annotation of one or more fundus images according to an embodiment of the disclosure. In some examples, the fundus of the human eye 110 refers to tissue in the posterior portion of the eyeball, which may include the inner membrane, retina, macula, and blood vessels of the eyeball. In some examples, a fundus image of a fundus of human eyes 110 may be obtained by the acquisition device 120. In some examples, the acquisition device 120 may include, but is not limited to, a camera or the like. The camera may be, for example, a color fundus camera. In some examples, the computerized quality control method is performed by a quality control system 300 (not shown; see FIG. 8) which can be applied to the use scenario 100.

In the use scenario 100, a plurality of human eyes 110 are photographed by one or more acquisition devices 120 to create a plurality of fundus images. Each of the plurality of fundus images are subsequently transmitted or otherwise transferred to a plurality of first annotation doctors A, which review and annotate each fundus image on one or more computer systems to obtain a plurality of doctor annotation results 130 for each of the images. In the example shown, first annotations doctors A comprises three annotation doctors: A1, A2, and A3; however, the plurality of annotation doctors A can be any number of doctors. After the plurality of doctor annotation results 130 are obtained, each of the plurality of doctor annotation results 130 for the image is analyzed against preset conditions (described later) and may be used as a target annotation result 140 (described later) based on this analysis. For example, as shown in FIG. 1, assuming that the doctor annotation results 130 of doctors A2 and A3 satisfy the preset conditions, the doctor annotation results 130 of doctors A2 and A3 may be included in the target annotation results 140 of that image. Any doctor annotation results 130 which fail to satisfy the preset conditions are not included in the target annotation results 140 by default. After all the target annotation results 140 are gathered, they are compiled into a final annotation result 150 (described later).

In some examples, a doctor annotation result 130 which fails to satisfy the preset conditions may be transmitted or otherwise transferred to a second annotation doctor B for re-annotation. Second annotation doctor B may be one or more doctors using one or more computer systems connected via quality control system 300. The reannotated results from the second annotation doctor B may be immediately included in the target annotation results 140 or may be checked against the preset conditions for a second time. If the annotated result fails to satisfy the preset conditions, the annotated results may be filtered out or may be transmitted to additional annotation doctors for continued re-annotation until the re-annotated result satisfies the preset condition such that it can be included in the target annotation results 140. In some examples, the second annotation doctor B may be a different doctor from those in the first annotation doctors A, while in other examples the second annotation doctor B may be a doctor from the first annotation doctors A. The first annotation doctors A and the second annotation doctor(s) B may include, but are not limited to, a professional ophthalmologist or an experienced doctor.

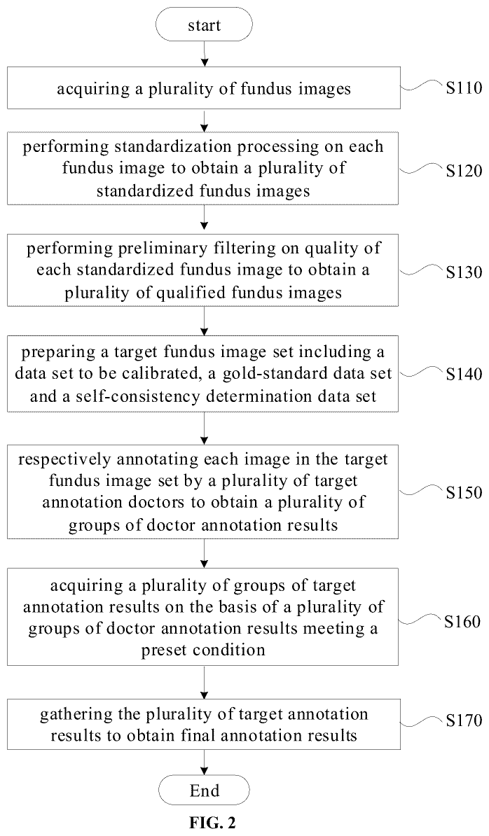

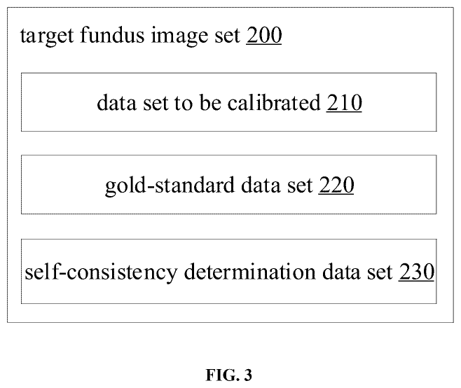

Hereinafter, a computerized quality control method according to the present disclosure will be described in detail with reference to the accompanying drawings. FIG. 2 is a flowchart illustrating the quality control method for data annotation on a fundus image according to an embodiment of the disclosure. In some examples, as shown in FIG. 2, the computerized quality control method may include acquiring a plurality of fundus images (step S110), performing standardization processing on each fundus image to obtain a plurality of standardized fundus images (step S120), performing preliminary filtering on quality of each standardized fundus image to obtain a plurality of qualified fundus images (step S130), preparing a target fundus image set including a data set to be calibrated (see FIG. 3), a gold-standard data set and a self-consistency determination data set (step S140), annotating each image in the target fundus image set by a plurality of first annotation doctors respectively to obtain a plurality of groups of doctor annotation results (step S150), acquiring a plurality of groups of target annotation results based on the plurality of groups of doctor annotation results meeting a preset condition (step S160), and gathering the plurality of groups of target annotation results to obtain a final annotation result (step S170). In this case, based on the gold-standard data set and the self-consistency determination data set, the doctor annotation result of the first annotation doctor that satisfies the preset conditions may be obtained as the target annotation result and gathered. Thus, the accuracy of data annotation of the fundus image may be improved. In some examples, the computerized quality control method is performed by quality control system 300 (not shown; see FIG. 8) and each of the steps S110, S120, S130, S140, S150, S160, and S170 may be performed by one or more software modules (accompanied by human input) which are stored in one or more non-transitory computer storage systems/servers and are executed by one or more computer processors connected via a computer network.

In some examples, in step S110, a plurality of fundus images may be acquired. In some examples, the fundus image may be a color fundus image. The color fundus images may clearly show the rich fundus information such as optic disc, optic cup, macula blood vessels, etc. In addition, the fundus image may be an RGB mode or a grayscale mode image or the like. In some examples, the fundus images may be fundus images acquired by the acquisition device 120. In other examples, the fundus images may be pre-stored in the server of quality control system 300. In some examples, quality control system 300 is configured to acquire and initiate the quality control method on up to 200,000 fundus images from a cooperative hospital with patient information removed.

In some examples, in step S120, standardization processing is performed on each fundus image, modifying each image's digital data structure, in order to obtain a plurality of standardized fundus images. In some examples the standardization process is configured to standardize the up to 200,000 fundus images sequentially or in parallel in a single operation performed by a computer processor. In some examples, the standardization processing S120 analyzes each fundus image to identify image data for modification. In some examples, the analysis of each fundus image comprises analyzing each image down to the pixel level and/or analyzing the digital data structures which make up the image. In some examples, the standardization processing may include any of the following: classifying the fundus image per the patient, unifying a name format of the fundus images, filtering out a non-fundus image, unifying a picture format of the fundus images (e.g., converting to jpg format), and unifying a background of the fundus images. Classifying the fundus image per the patient may comprise encoding confidential data of the fundus image into a coded hash value. Unifying a name format of the fundus images may comprise removing patient information in the name of the fundus image and standardizing the name of the fundus image. Filtering out non-fundus images may comprise identifying non-fundus images, which may include, but is not limited to, a fundus mosaic, an anterior segment map, or an image other than a 45-degree fundus image centered on the optic disc and macula. Unifying a background of the fundus images may comprise modifying the fundus images to have a uniform background color (e.g., black).

In some examples, in step S130, the quality of each of the standardized fundus images may be preliminarily filtered to obtain a plurality of qualified fundus images.

In some examples, the preliminary filtering may include classifying the standardized fundus images into at least two image quality grades including qualified and unqualified. Thereby, the quality of the standardized fundus images may be preliminarily filtered to quickly acquire qualified fundus images.

In some examples, the quality of the standardized fundus image may be determined by the plurality of first annotation doctors A to classify the standardized fundus image into a plurality of image quality grades. In some examples, the standardized fundus image may be ranked based on factors that affect the quality of the fundus image. In some examples, factors affecting the quality of the fundus image may include, but are not limited to, at least one of a location at which the fundus image was taken, an exposure of the fundus image, and a definition of the fundus image. For example, a standardized fundus image with an acceptable image quality grade may be an image with the correct location, moderate exposure, and good definition. In this case, the quality of the standardized fundus image is ranked. Thus, it is facilitated to obtain a qualified fundus image.

However, the examples of the present disclosure are not limited hereto, in other examples, the standardized fundus images may be classified in more precise way in the preliminary filtering. For example, the standardized fundus image may be classified into at least five image quality grades. In some examples, the five image quality grades may include very good, good, average, poor, and very poor. In some examples, the image quality grade may also include unreadable images caused by abnormalities in the shot region (e.g., non-fundus images), no image or image acquisition technique issues, and other issues. In some examples, the image quality grades may be qualified, barely qualified, and unqualified.

Additionally, in some examples, in step S130, a plurality of qualified fundus images may be acquired. In some examples, the qualified fundus image may be a standardized fundus image with a qualified image quality grade. However, the examples of the present disclosure are not limited thereto, in other examples, a qualified fundus image may be a standardized fundus image with image quality grades of very good, good, average, and poor. The qualified fundus image may be a standardized fundus image of which image quality grade is very good, good and average or the qualified fundus image may be a standardized fundus image of which image quality grade is very good and good. In other examples, a qualified fundus image may be a standardized fundus image with an image quality grade of qualified and barely qualified. Thus, a qualified fundus image may be obtained.

FIG. 3 is a block diagram illustrating a target fundus image set according to an embodiment of the disclosure. As described above, the quality control method may include step S140 (see FIG. 2). In some examples, in step S140, a target fundus image set 200 including a data set to be calibrated 210, a gold-standard data set 220, and a self-consistency determination data set 230 may be prepared. As shown in FIG. 3, in some examples, the target fundus image set 200 may include a data set to be calibrated 210, a gold-standard data set 220, and a self-consistency determination data set 230.

In some examples, the data set to be calibrated 210 may include a plurality of qualified fundus images. In some examples, the data set to be calibrated 210 may include all qualified fundus images obtained in step S130. In some examples, the data set to be calibrated 210 may include the partial qualified fundus images obtained in step S130. In some examples, all qualified fundus images obtained in step S130 may be grouped and each group of qualified fundus images may be taken as one data set to be calibrated 210. For example, all of the qualified fundus images obtained in step S130 may be grouped in a group of 80, 90 or 100 images.

Additionally, in some examples, gold-standard data set 220 may include a first preset number of gold-standard fundus images. The gold-standard fundus image may be a fundus image for which correct annotation results are known. In some examples, the gold-standard fundus image may be a fundus image of a known correct annotation result from an annotation database. In some examples, the first preset number may be 5 to 20. For example, the first preset number may be 5, 10, 15, or 20, etc. However, the examples of the present disclosure are not limited thereto, and in other examples, the first preset number may be other values.

Additionally, in some examples, the self-consistency determination data set 230 may consist of images in the data set to be calibrated 210. In some examples, the number of images in the self-consistency determination data set 230 may be at least one. In some examples, the number of images in the self-consistency determination data set 230 may be 5 to 20. For example, the number of images in the self-consistency determination data set 230 may be 5, 10, 15, or 20, etc. However, the examples of the present disclosure are not limited hereto, in other examples, the number of images in the self-consistency determination data set 230 may be other values. In some examples, the number of images in the self-consistency determination data set 230 may be less than the number of images in the data set to be calibrated 210. Thus, the image in the self-consistency determination data set 230 may be repeated with part of the images in the data set to be calibrated 210. Additionally, in some examples, each image of the target fundus image set 200 may serve as each target fundus image.

In some examples, in step S150, each image in the target fundus image set 200 may be annotated by a plurality of first annotation doctors A to obtain a plurality of groups of doctor annotation results 130. For example, assuming that three first annotation doctors A annotate the target fundus image set 200 respectively, three first annotation doctors A may obtain three groups of doctor annotation results 130. In some examples, a plurality of first annotation doctors A may annotate each image in the target fundus image set 200 using an online annotation system. In some examples, the number of first annotation doctors A may be greater than or equal to three. For example, the number of first annotation doctors A may be 3, 5, 7, or 9, etc.

In some examples, the doctor annotation results 130 for each image in the target fundus image set 200 may include at least one determination result. In some examples, the determination result may include disease information of no obvious abnormality or of a disease. In some examples, if there is not any disease in the image of the target fundus image set 200, the doctor annotation result 130 for that image may be no obvious abnormality. In some examples, the doctor annotation result 130 may be a determination result of multiple diseases. For example, the doctor annotation result 130 may be diabetic retinopathy stage I and the presence of glaucoma.

In some examples, the doctor annotation result 130 may include eye difference (e.g., left or right eye) and an image quality grade of the quality of each image in the target fundus image set 200. In some examples, if the quality of each standardized fundus image is not classified in more precise way in the preliminary filtering, the first annotation doctor A may classify the quality of each image in the target fundus image set 200 in more precise way in the annotation process. In other examples, if the quality of each standardized fundus image is classified in more precise way in the preliminary filtering, the first annotation doctor A may re-classify the quality of each image in the target fundus image set 200 in the annotation process. Specific contents are described with reference to a more detailed classification of the standardized fundus image. In this case, the final annotation result 150 may be subsequently determined in conjunction with a more detailed image quality grade. In some examples, the image quality grades in the doctor annotation result 130 may include image quality grades obtained by the preliminary filtering and image quality grades obtained in the annotation process.

In some examples, the disease may include at least one of diabetic retinopathy, hypertensive retinopathy, glaucoma, retinal vein occlusion, retinal artery occlusion, age-related macular degeneration, high myopia macular degeneration, retinal detachment, optic nerve disease, congenital abnormalities of disc development. Thereby, at least one disease may be annotated. However, the examples of the present disclosure are not limited thereto, and the quality control method of the present disclosure may be easily generalized to quality control of data annotation of other diseases or data annotation of other fields. In some examples, the disease information may be a staging based on the severity of the disease. For example, diabetic retinopathy may be staged as stage I, II, III, IV, V, and VI. In other examples, the disease information may be the presence of a certain disease, e.g., the disease information may be the presence of glaucoma.

As described above, the quality control method may include step S160 (see FIG. 2). In some examples, in step S160, a plurality of groups of target annotation results 140 may be obtained based on a plurality of groups of doctor annotation results 130 meeting a preset condition. In some examples, in step S160, the self-consistency and gold-standard consistency of the corresponding first annotation doctor A may be calculated based on the doctor annotation result 130.

As described above, each image of the target fundus image set 200 may serve as each target fundus image. In some examples, self-consistency may be obtained by determining whether the doctor annotation results 130 obtained by each first annotation doctor A annotating the same target fundus image twice are consistent or not. In some examples, it may be illustrated that the higher the self-consistency, the more stable the annotation level of the first annotation doctor A may be. Specifically, in some examples, in calculating self-consistency, the doctor annotation results 130 of each image in the self-consistency determination data set 230 may be obtained, as well as the doctor annotation results 130 of images in the data set to be calibrated 210 that are repeated with respective image in the self-consistency determination data set 230. In some examples, it is to take any one of the two groups of annotation results as a first group of annotation results and the other set as a second group of annotation results and evaluate using a self-consistency determination and evaluation method to obtain self-consistency.

In some examples, the self-consistency determination method may use a quadratic weighted kappa coefficient to calculate the disease self-consistency of each of the first annotation doctors A for each of the diseases. In some examples, the quadratic weighted kappa coefficient κ for a single disease may be

$$κ = 1 - \frac{{\sum }_{i , j} {W}_{ij} {X}_{ij}}{{\sum }_{i , j} {W}_{ij} {E}_{ij}}$$

Here, Wᵢⱼ may represent a quadratic weighting coefficient, Xᵢⱼ may represent a number of target fundus images in which the determination result in the first group of annotation results is i and the determination result in the second group of annotation results is j, Ei may represent an expected number of target fundus images in which the determination result in the first group of annotation results is i and the determination result in the second group of annotation results is j may be represented. In some examples, when i is not equal to j, Eᵢⱼ may be zero. In some examples, a quadratic weighting factor Wᵢⱼ may be set as needed to highlight the importance of a certain determination result. Thus, it is able to check consistency between the first group of annotation results and the second group of annotation results.

In some examples, in the self-consistency determination method, self-consistency of each disease may be weighted to calculate the self-consistency of each first annotation doctor A. For example, the weight for disease self-consistency of diabetic retinopathy may be set to 1, and the weight for disease self-consistency of other diseases may be set to 0.5. Thus, the self-consistency of each first annotation doctor A may be calculated based on the self-consistency determination method. However, the examples of the present disclosure are not limited hereto, and in other examples, self-consistency may be calculated in other ways.

As described above, in step S160, the gold-standard consistency of the corresponding first annotation doctor A may be calculated based on the doctor annotation result 130. Specifically, in some examples, in calculating the gold-standard consistency, the correct annotation result for the gold-standard data set 220 may be taken as the first group of annotation results, that is, the gold-standard fundus image is taken as the first group of annotation results, and the doctor annotation results 130 for each image in the gold-standard data set 220 may be taken as the second set of annotation results. In some examples, the gold-standard consistency may be obtained based on the first group of annotation results and the second group of annotation results and evaluated using a gold-standard consistency determination and evaluation method.

In some examples, the gold-standard consistency determination method may use a quadratic weighted kappa coefficient to calculate the disease gold-standard consistency for each first annotation doctor A to determine each disease. In some examples, gold-standard consistency of each disease may be weighted to calculate a gold-standard consistency for each first annotation doctor A. Thus, the self-consistency of each first annotation doctor A may be calculated based on the gold-standard consistency determination method. The detailed description of the self-consistency determination method may be referenced for specific contents. However, the examples of the present disclosure are not limited hereto, and in other examples, the gold-standard consistency may be calculated in other ways.

In some examples, in step S160, the doctor annotation result 130 of the first annotation doctor A meeting the preset condition may be obtained and the doctor annotation result 130 may be taken as the target annotation result 140. In some examples, the preset condition may be d_{self}≤D and d_{gold}≤D Here, d_{self} is a self-evaluation index based on self-consistency, d_{gold} is a gold-standard evaluation index based on gold-standard consistency, and Dis an evaluation index threshold value. In some examples, D≤5%. Thus, the preset condition may be determined based on the evaluation index threshold value.

In some examples, the self-evaluation index d_{self} may satisfy the formula: d_{self}=|J_{self}−κ_{self}|/κ_{self}×100% Here, J_{self}=SE_{self}+SP_{self}−1, SE_{self} is a sensitivity of the first, annotation doctor A obtained based on the two groups of annotation results for evaluating self-consistency, SP_{self} is the specificity of the first annotation doctor A obtained based on the two groups of annotation results for evaluating self-consistency, κ_{self} is self-consistency of the first annotation doctor A. In some examples, any group of the two groups of annotation results used to evaluate self-consistency may be taken as a gold-standard to evaluate the other group to obtain the sensitivity and specificity of the first annotation doctor A.

In some examples, the gold-standard evaluation index d_{gold} may satisfy the formula: d_{gold}=|J_{gold}−κ_{gold}|/κ_{gold}×100% Here, J_{gold}=SE_{gold}+SP_{gold}−1, SE_{gold} is a sensitivity of the first annotation doctor A obtained based on the two groups of annotation results for evaluating the gold-standard consistency, and SP gold is a specificity of the first annotation doctor A obtained based on the two groups of annotation results for evaluating the gold-standard consistency, K_{gold} is the gold-standard consistency of the first annotation doctor A. In some examples, the first group of the two groups of annotation results used to evaluate gold-standard consistency may be taken as the gold-standard to evaluate the second group of annotation results to obtain the sensitivity and specificity of the first annotation doctor A.

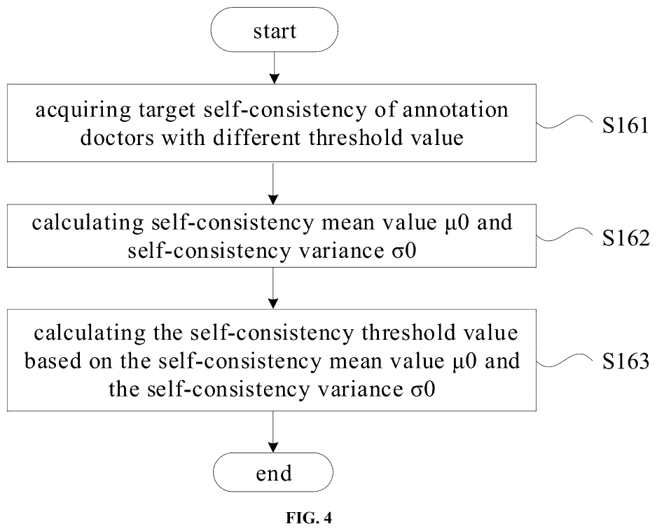

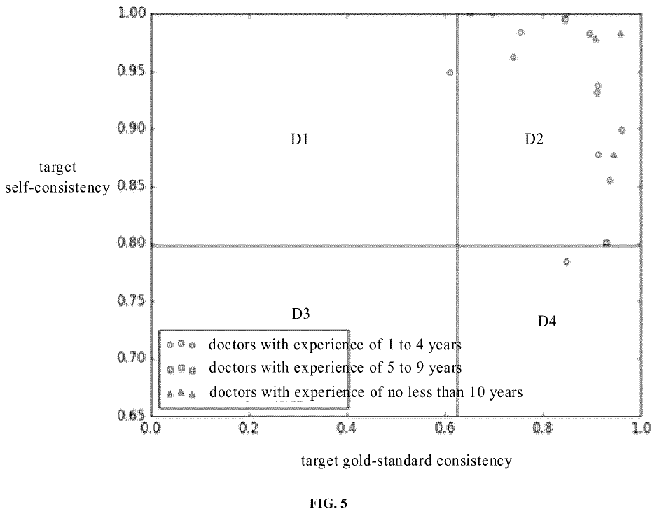

FIG. 4 is a flowchart illustrating the determination of a self-consistency threshold value according to an embodiment of the disclosure. FIG. 5 is a statistical chart illustrating target self-consistency and target gold-standard consistency according to an embodiment of the disclosure. The first region D1, the second region D2, the third region D3, and the fourth region D4 are four regions in the statistical chart. In some examples, the preset condition may be that the self-consistency is greater than the self-consistency threshold value and the gold-standard consistency is greater than the gold-standard consistency threshold value. In some examples, it may be to analyze the target self-consistency and target gold-standard consistency of doctors with different threshold value annotation and use abnormality detection to determine the self-consistency threshold value and gold-standard consistency threshold value. Thus, the self-consistency threshold value and the gold-standard consistency threshold value may be determined.

In some examples, as shown in FIG. 4, the process of determining a self-consistency threshold value based on an abnormality detection approach may include obtaining target self-consistency for annotation doctors with different threshold value (step S161), calculating a self-consistency mean value μ₀ and a self-consistency variance σ₀ (step S162), and calculating a self-consistency threshold value based on the self-consistency mean po and the self-consistency variance σ₀ (step S163). Thus, the self-consistency threshold value may be determined.

In some examples, in step S161, target self-consistency for annotation doctors with different threshold values may be obtained. Specifically, the target self-consistency of annotation doctors with different threshold values may be analyzed. For example, the target self-consistency of a threshold value annotation doctor with an experience of 1-4 years of a threshold value annotation doctor with an experience of 5-9 years, and of a threshold value annotation doctor with an experience of no less than 10 years may be analyzed. In some examples, the target self-consistency and the target gold-standard consistency (described later) may be obtained simultaneously. As an example of the target self-consistency and target gold-standard consistency statistics, FIG. 5 illustrates the statistic results of target self-consistency and target gold-standard consistency for threshold value annotation doctors with different seniority. The circle may represent the target self-consistency and target gold-standard consistency of the threshold value annotation doctor with an experience of 1-4 years. The square may represent the target self-consistency and target gold-standard consistency of the threshold value annotation doctor with an experience of 5-9 years. The triangle may represent the target self-consistency and target gold-standard consistency of the threshold value annotation doctor with an experience of no less than ten years. The first region D1, the second region D2, the third region D3, and the fourth region D4 are four regions in the statistical chart. It may be seen from FIG. 5 that the statistical results of the target self-consistency and the target gold-standard consistency of threshold value annotation doctors with an experience of 1-4 years fall into the first region D1 and the fourth region D4, while the statistical results of the target self-consistency and the target gold-standard consistency of the threshold value annotation doctors with an experience of no less than 5 years mainly fall into the second region D2.

In some examples, in step S162, a self-consistency mean value μ₀ and a self-consistency variance σ₀ may be calculated. In some examples, the self-consistency mean value μ₀ and the self-consistency variance σ of the target self-consistency may be calculated.

In some examples, in step S163, a self-consistency threshold value may be calculated based on the self-consistency mean value μ₀ and the self-consistency variance σ₀. Specifically, in some examples, the self-consistency threshold value may be μ₀−1.96×σ₀ under the assumption that the target self-consistency satisfies a Gaussian distribution. In this case, the probability of an anomaly occurring is less than 2.5%. In some examples, the self-consistency threshold value may be 0.7977.

In some examples, the process of determining the gold-standard consistency threshold value based on the manner of abnormality detection may include obtaining target gold-standard consistency of annotation doctors with different threshold values, calculating a gold-standard consistency mean value μ₁ and a gold-standard consistency variance σ₁ of the target gold-standard consistency, and calculating the gold-standard consistency threshold value based on the gold-standard consistency mean value μ₁ and the gold-standard consistency variance σ₁. Thus, the gold-standard consistency threshold value may be determined. In some examples, the gold-standard uniformity threshold value may be μ₁−1.96×σ₁ under the assumption that the target gold-standard uniformity satisfies a Gaussian distribution. In this case, the probability of an anomaly occurring is less than 2.5%. In some examples, the gold-standard consistency threshold value may be 0.6235. A detailed description of the process for determining the gold-standard consistency threshold value may be found in the process for determining the self-consistency threshold value for reference and will not be described in detail herein.

However, the examples of the present disclosure are not limited hereto, in other examples, abnormality detection in other way may be used to determine the self-consistency threshold value and the gold-standard consistency threshold value.

In some examples, in step S160, the doctor annotation result 130 of the first annotation doctor A which does not meet the preset condition may be re-annotated by the second annotation doctor B for each image in the target fundus image set 200. In some examples, the doctor annotation result 130 that does not meet the preset condition may be continually re-annotated until the doctor annotation result 130 that meets the preset condition is obtained as the target annotation result 140. In this case, the doctor annotation result 130 of the first annotation doctor A which does not meet the preset condition is re-annotated. Thus, the target annotation result 140 may be obtained. In some examples, the second annotation doctor may be different from the first annotation doctor in step S150.

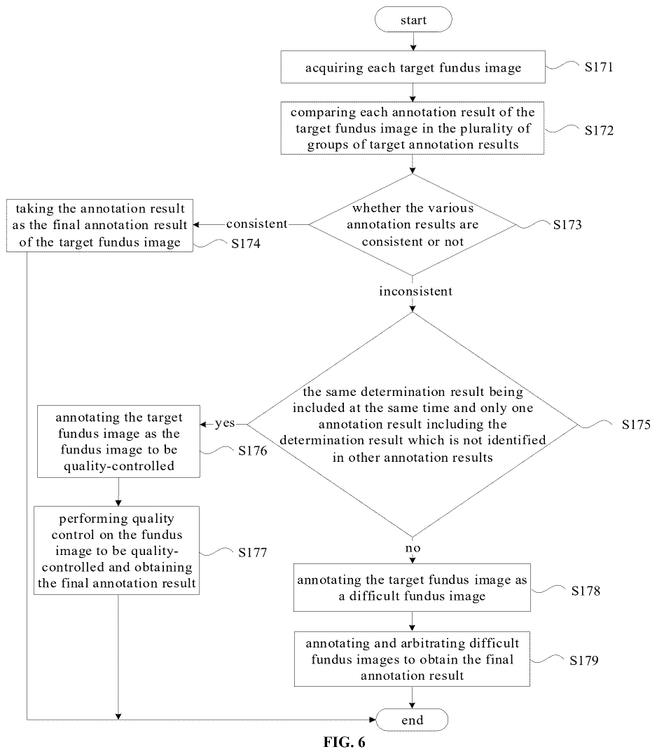

FIG. 6 is a flowchart illustrating the manner in which the examples of the present disclosure are gathered. As described above, the quality control method may include step S170 (see FIG. 2). In some examples, in step S170, a plurality groups of target annotation results 140 may be gathered to obtain a final annotation result 150.

In some examples, absolute majority voting is used to compare each annotation result of each target fundus image in the plurality of groups of target annotation results 140 to determine a final annotation result 150 for each target fundus image. Specifically, when individual annotation results are compared to each other using Absolute Majority Voting, the annotation results will be accepted as part of the final annotation result 150 if more than half of determination results of the annotation results are consistent (that is, more than half of the valid votes are required to be accepted). In some examples, if the final annotation result 150 cannot be determined (i.e., the number of valid votes is not more than half), the target fundus image is annotated as a difficult fundus image. In some examples, difficult fundus images may be annotated and arbitrated to obtain a final annotation result 150. Thus, the final annotation result 150 may be obtained based on the absolute majority voting method.

In some examples, difficult fundus images may be annotated by an arbitration doctor to obtain an arbitration annotation result. In some examples, the arbitration annotation result may include at least one determination result. In some examples, the arbitration annotation result may be taken as the final annotation result 150.

However, the examples of the present disclosure are not limited thereto, in other examples, as shown in FIG. 6, the process of the gathered manner of step S170 may include steps S171 to S179. In this case, by comparing the respective annotation results of the respective target fundus images in the plurality of groups of target annotation results 140, the target fundus image may be divided into a target fundus image having a final annotation result 150, a fundus image to be quality-controlled, and a difficult fundus image, and the final annotation result 150 may be obtained.

In some examples, in step S171, each target fundus image may be acquired. Specifically, in some examples, each target fundus image in the target fundus image set 200 may be traversed sequentially and compared in step S172.

In some examples, in step S172, the respective annotation results of the respective target fundus images in the plurality of sets of target annotation results 140 obtained in step S171 may be compared. For example, assuming there are three groups of target annotation results 140, each target fundus image may have three annotation results originated from each group of target annotation results 140.

In some examples, in step S173, it may be determined whether the respective annotation results are consistent. For example, it is able to compare the three annotation results of step S172 and make sure whether they are identical or not.

In some examples, if the respective annotation results are consistent, the process may proceed to step S174. In some examples, in step S174, the annotation result may be taken as the final annotation result 150 of the target fundus image. In some examples, it may be determined that the respective annotation results are consistent when the determination results included in the respective annotation results are totally identical. For example, if there is no obvious abnormality in each annotation result, it may be determined that each annotation result is consistent. For another example, if each annotation result is stage I diabetic retinopathy and glaucoma is present, it may be determined that each annotation result is consistent.

In some examples, if the plurality of annotation results are inconsistent, step S175 may be entered. In some examples, in step S175, it may be determined whether each annotation result includes the same determination result at the same time and only one annotation result includes a determination result which is not identified in other annotation results, in case of “yes”, step S176 may be entered, otherwise, step S177 may be entered.

For example, assume that the plurality of annotation results of the target fundus image are a first annotation result, a second annotation result, and a third annotation result, respectively. The first annotation result is diabetic retinopathy stage I, the second annotation result is diabetic retinopathy stage I, and the third annotation result is diabetic retinopathy stage I and presence of glaucoma. In this case, diabetic retinopathy stage I is the same determination result included in each annotation result at the same time. Presence of glaucoma is an unidentified determination result, and only one annotation result includes presence of glaucoma. However, the examples of the present disclosure are not limited to hereto, and in other examples, the determination may be made by other determination conditions. For example, the condition that each annotation result may include the same determination result at the same time, and at least one annotation result may include a determination result that is not recognized in the other annotation results is taken as the determination condition in step S175.

In some examples, in step S176, the target fundus image may be annotated as a fundus image to be quality controlled.

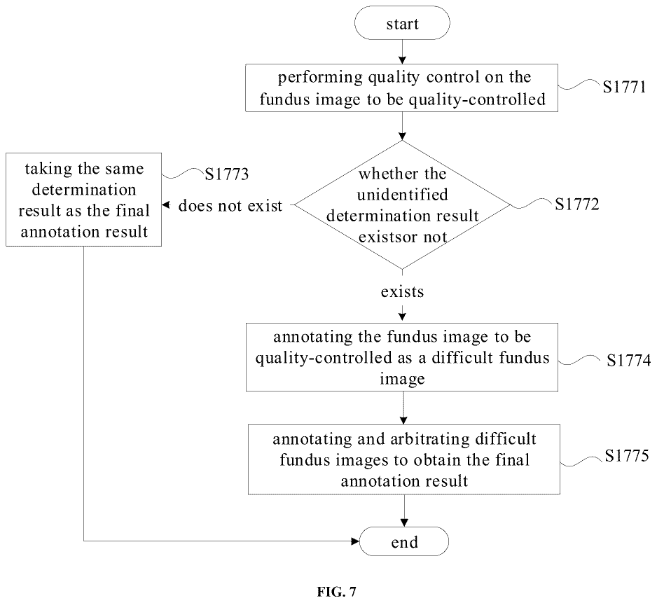

FIG. 7 is a flowchart illustrating computerized quality control of a fundus image and obtaining a final annotation result according to an embodiment of the disclosure. In some examples, in step S177, the fundus image to be quality-controlled may be quality-controlled and a final annotation result 150 is obtained. As shown in FIG. 7, in some examples, the process of quality-controlling the fundus image to be quality-controlled and obtaining the final annotation result may include steps S1771 to S1775.

In some examples, in step S1771, quality control may be performed on the fundus image to be quality-controlled. In some examples, the fundus image to be quality-controlled may be quality-controlled by a quality control doctor to obtain a quality control determination result. In some examples, the unidentified determination result in the fundus image to be quality-controlled (for example, only one annotation result described in step S175 includes the presence of glaucoma) may be evaluated to obtain the quality control determination result (for example, there are unrecognized determination results or there are no unidentified determination results).

In some examples, in step S1772, it may be identified whether unidentified determination result exists or not based on the quality control determination result of step S1771, in case of “no”, step S1773 may be entered, otherwise, step S1774 may be entered.

In some examples, in step S1773, the same determination result is taken as the final annotation result 150 in step S1773. For example, assuming that the plurality of annotation results of the target fundus image are a first annotation result, a second annotation result, and a third annotation result, respectively. Here, the first annotation result is diabetic retinopathy stage I, the second annotation result is diabetic retinopathy stage I, the third annotation result is diabetic retinopathy stage I with presence of glaucoma. In this case, the diabetic retinopathy stage I is the same determination result that each annotation result simultaneously includes, and may be taken as the final annotation result 150 of the target fundus image.

In some examples, in step S1774, the fundus image to be quality-controlled may be annotated as a difficult fundus image.

In some examples, in step S1775, difficult fundus images may be annotated and arbitrated to obtain a final annotation result 150. In some examples, difficult fundus images may be annotated by an arbitration doctor to obtain an arbitration annotation result. In some examples, the arbitration annotation result may include at least one determination result. In some examples, the arbitration annotation result may be taken as the final annotation result 150. In some examples, a final annotation result 150 may be obtained based on a plurality of target annotation results 140, quality control determination results, and arbitration annotation results for a difficult fundus image.

As described above, the process of the gathering of step S170 may include step S178. In some examples, in step S178, the target fundus image may be marked as a difficult fundus image.

In some examples, in step S179, difficult fundus images may be annotated and arbitrated to obtain a final annotation knot 150. In some examples, a final annotation result 150 may be obtained based on a plurality of target annotation results 140 and arbitration annotation results for a difficult fundus image. Thus, the final annotation result 150 of the difficult fundus image may be obtained. The detailed content may be seen in relevant description in step S1775.

In some examples, the doctor annotation results 130 of the target fundus image set 200 may be counted to obtain statistical results. In some examples, the statistical results may include a gold-standard consistency re-annotation ratio. In some examples, the gold-standard consistency re-annotation ratio may be the ratio of the re-annotated target fundus image in the target fundus image set 200 due to the unqualified gold-standard consistency. In some examples, the statistical results may include a self-consistency re-annotation ratio. In some examples, self-consistency re-annotation ratio may be the ratio of the re-annotated target fundus images in the target fundus image set 200 due to unqualified self-consistency.

In some examples, the annotation process may be quality-controlled based on statistical results. For example, if the gold-standard consistency re-annotation ratio exceeds a pre-set value, the assignment of annotation tasks to relevant annotation doctors may be subsequently reduced or cancelled.

In some examples, annotation reports may be output. In some examples, the annotation report may include at least one of a doctor annotation result 130, a target annotation result 140, a final annotation result 150, a quality control determination result, an arbitration annotation result, and a statistical result.

Hereinafter, the quality control system 300 for data annotation on a fundus image according to the present disclosure will be described in detail with reference to FIG. 8. The quality control system 300 for data annotation on a fundus image in the present disclosure may sometimes be referred to simply as “quality control system 300”. The quality control system 300 is comprised of one or more interconnected computer systems and which execute the quality control method. FIG. 8 is a block diagram illustrating the quality control system 300 and its associated software modules 310, 320, 330, 340, 350, 360, and 370. The term software modules, as used herein, refers to separate software processes which are executed via one or more computer processors to perform associated steps. The software modules may be stored as separate software programs or as executable processes in a single software program.

In some examples, as shown in FIG. 8, the quality control system 300's software modules comprise: an acquisition module 310, a standardization processing module 320, a preliminary filtering module 330, a data preparation module 340, an annotation module 350, an evaluation module 360, and a gathering module 370. These modules 310, 320, 330, 340, 360, and 370 each execute processes corresponding to steps S110, S120, S130, S140, S150, S160, and S170 of FIG. 2 respectively. The acquisition module 310 may be used to acquire a plurality of fundus images, by performing a method like step S110. The standardization processing module 320 may be used to normalize each fundus image to obtain a plurality of standardized fundus images, by performing a method like S120. The preliminary filtering module 330 may be used to perform preliminary filtering on quality of each of the standardized fundus images to obtain a plurality of qualified fundus images, by performing a method like S130. The data preparation module 340 may be used to prepare the target fundus image set 200 including the data set to be calibrated 210, the gold-standard data set 220, and the self-consistency determination data set 230, by performing a method like S140. The annotation module 350 may be used to obtain a plurality of groups of doctor annotation results 130 for each image in the target fundus image set by a plurality of first annotation doctors A, respectively, by performing a method like S150. The evaluation module 360 may be used to obtain a plurality of sets of target annotation results 140 meeting preset condition based on a plurality of groups of doctor annotation results, by performing a method like S160. The gathering module 370 may be used to gather a plurality of groups of target annotation results 140 to obtain a final annotation result 150, by performing a method like S170. In this case, the doctor annotation result 130 of the first annotation doctor A meeting the preset condition may be acquired as the target annotation result 140 and gathered based on the gold-standard data set and the self-consistency determination data set. Thus, the accuracy of data annotation of the fundus image may be improved.

In some examples, in the acquisition module 310, the fundus image may be a color fundus image. The color fundus images may clearly show the rich fundus information such as optic disc, optic cup, macula blood vessels. The detailed description may refer to the relevant description of step S110, which will not be repeated here.

In some examples, in the standardization processing module 320, in some examples, the standardization processing may include at least one of classifying the fundus images per patient, unifying a name format of the fundus image, filtering out a non-fundus image, unifying a picture format of the fundus image, and unifying a background of the fundus image. Thus, the standardization processing on fundus images may be performed. The detailed description may refer to the relevant description of step S120, which will not be repeated here.

In some examples, in the preliminary filtering module 330, in some examples, the preliminary filtering may classify the standardized fundus image into at least two image quality grades including qualified and unqualified. Thereby, the quality of the standardized fundus images may be preliminarily filtered quickly to acquire qualified fundus images. In some examples, the qualified fundus image may be a standardized fundus image with a qualified image quality grade. Thus, a qualified fundus image may be obtained. However, the examples of the present disclosure are not limited thereto, in other examples, the standardized fundus images may be classified in more precise way in the preliminary filtering. The detailed description may refer to the relevant description of step S130, which will not be repeated here.

In some examples, in the data preparation module 340, the target fundus image set may include the data set to be calibrated 210, the gold-standard data set 220, and the self-consistency determination data set 230. In some examples, the data set to be calibrated 210 may include a plurality of qualified fundus images. In some examples, gold-standard data set 220 may include a first preset number of gold-standard fundus images. The gold-standard fundus image may be a fundus image for which correct annotation results are known. In some examples, the self-consistency determination data set 230 may consist of images in the data set to be calibrated 210. In some examples, the number of images in the self-consistency determination data set 230 may be at least one. In some examples, each image of the target fundus image set 200 may be taken as each target fundus image. The detailed description may refer to the relevant description of step S140, which will not be repeated here.

In some examples, in the annotation module 350, the doctor annotation results 130 for each image in the target fundus image set 200 may include at least one determination result. In some examples, the determination result may include disease information which is of no abnormality or a disease. In some examples, the disease may include at least one of diabetic retinopathy, hypertensive retinopathy, glaucoma, retinal vein occlusion, retinal artery occlusion, age-related macular degeneration, high myopia macular degeneration, retinal detachment, optic nerve disease, congenital abnormalities of disc development. Thereby, at least one disease may be annotated. In some examples, in the annotation, the image quality of each image in the target fundus image set may be further classified into five image quality grades including very good, good, average, poor, and very poor. In this case, the final annotation result 150 may subsequently be determined in conjunction with a more precise image quality grade. The detailed description may refer to the relevant description of step S150, which will not be repeated here.

In some examples, in the evaluation module 360, the doctor annotation result 130 of the first annotation doctor A meeting the self-consistency and gold-standard consistency requirements may be obtained and taken as the target annotation result 140. In some examples, the preset condition may be a doctor annotation result 130 of a first annotation doctor A having a self-consistency greater than a self-consistency threshold value and a gold-standard consistency greater than a gold-standard consistency threshold value. Thus, the preset condition may be determined based on the self-consistency threshold value and the gold-standard consistency threshold value. In some examples, the preset condition may be d_{self}≤D and d_{gold}≤D Here, d_{self} is a self-evaluation index based on self-consistency, d_{gold} is a gold-standard evaluation index based on gold-standard consistency, and Dis an evaluation index threshold value. In some examples, D≤5% Thus, the preset condition may be determined based on the evaluation index threshold value. In some examples, the doctor annotation result 130 of the first annotation doctor A that does not meet the preset condition may be re-annotated by the second annotation doctor B for each image in the target fundus image set 200 until the doctor annotation result 130 meeting the preset condition is obtained as the target annotation result 140. The detailed description may refer to the relevant description of step S160, which will not be repeated here.

In some examples, in the evaluation module 360, the self-evaluation index d_{self} may satisfy the formula: d_{self}=| J_{self}−κ_{self}|/κ_{self}×100% Here, J_{self}=SE_{self}+SP_{self}−1, SE_{self} is the, sensitivity of the first annotation doctor A obtained based on the two groups of annotation results for evaluating self-consistency and SP_{self} is the specificity of the first annotation doctor A obtained based on the two groups of annotation results for evaluating self-consistency, κ_{self} is the self-consistency of the first annotation doctor A. In some examples, any one group of the two groups of annotation results used to evaluate self-consistency may be taken as a gold-standard to evaluate the other group to obtain the sensitivity and specificity of the first annotation doctor A.

In some examples, in the evaluation module 360, the gold-standard evaluation index d_{gold} may satisfy the formula: d_{gold}=|J_{gold}−κ_{gold}|/κ_{gold}×100% Here, J_{gold}=SE_{gold}+SP_{gold}−1, SE_{gold} is the sensitivity of the first annotation doctor A obtained based on the two groups of annotation results for assessing the gold-standard consistency, SP gold is the specificity of the first annotation doctor A obtained based on the two groups of annotation results for assessing the gold-standard consistency, κ_{gold} is the gold-standard consistency of the first annotation doctor A. In some examples, the first of the two groups of annotation results used to evaluate gold-standard consistency may be taken as the gold-standard to evaluate the second group of annotation results to obtain the sensitivity and specificity of the first annotation doctor A.

In some examples, in calculating self-consistency, the evaluation module 360 may obtain two groups of doctor annotation results 130 for each image in the self-consistency determination data set and for images in the data set to be calibrated 210 that are repeated with respective image in the self-consistency determination data set 230. In some examples, self-consistency may be obtained by taking any one of the two groups of annotation results as a first group of annotation results and the other set as a second group of annotation results and evaluating using a self-consistency determination and evaluation method. Thus, the self-consistency of each first annotation doctor A may be calculated based on the self-consistency determination method. The detailed description may refer to the relevant description of step S160, which will not be repeated here.

In some examples, in calculating the gold-standard consistency, the evaluation module 360 may take the correct annotation result for the gold-standard data set 220 as the first group of annotation results, that is, the annotation result for the gold-standard fundus image is taken as the first group of annotation results, and the doctor annotation result 130 for each image in the gold-standard data set 220 is taken as the second group of annotation results. In some examples, self-consistency may be obtained based on the first group of annotation results and the second group of annotation results and evaluated using a gold-standard consistency determination evaluation method. Thus, the self-consistency of each first annotation doctor A may be calculated based on the gold-standard consistency determination method. The detailed description may refer to the relevant description of step S160, which will not be repeated here.

In some examples, the self-consistency determination method in the evaluation module 360 may be using a quadratic weighted kappa coefficient to calculate the disease self-consistency for each of the first annotation doctors A determining each of the diseases. In some examples, the quadratic weighted kappa coefficient for a single disease may be

$$κ = 1 - \frac{{\sum }_{i , j} {W}_{ij} {X}_{ij}}{{\sum }_{i , j} {W}_{ij} {E}_{ij}}$$

Here, Wᵢⱼ may represent a quadratic weighting coefficient, Xᵢⱼ may represent the number of target fundus images in which the determination result in the first group of annotation results is i and the determination result in the second group of annotation results is j, Eᵢⱼ may represent the expected number of target fundus images in which the determination result in the first group of annotation results is i and the determination result in the second group of annotation results is j. In some examples, when i is not equal to j, Eᵢⱼ may be zero. In some examples, a quadratic weighting factor Wᵢⱼ may be set as needed to highlight the importance of a certain determination result. Thus, it is able to check consistency between the first group of annotation results and the second group of annotation results. In some examples, in the self-consistency determination method, each disease self-consistency may be weighted to calculate the self-consistency of each first annotation doctor A. The detailed description may refer to the relevant description of step S160, which will not be repeated here.

In some examples, the gold-standard consistency determination method in the evaluation module 360 may be using a quadratic weighted kappa coefficient to calculate the disease gold-standard consistency for each of the first annotation doctors A to determine each disease. In some examples, each disease gold-standard consistency may be weighted to calculate a gold-standard consistency for each first annotation doctor A. Thus, the self-consistency of each first annotation doctor A may be calculated based on the gold-standard consistency determination method. The detailed description may refer to the relevant description of step S160, which will not be repeated here.

In some examples, the evaluation module 360 may analyze the target self-consistency and target gold-standard consistency of different threshold value annotation doctors and determine the self-consistency threshold value and the gold-standard consistency threshold value in the manner of abnormality detection. Thus, the self-consistency threshold value and the gold-standard consistency threshold value may be determined. In some examples, the abnormality detection of the self-consistency threshold value may be performed in such a way as to obtain target self-consistency of different threshold value annotation doctors and to calculate a mean self-consistency μ₀ and a variance self-consistency co, and under the assumption that the target self-consistency satisfies a Gaussian distribution, the self-consistency threshold value may be μ₀−1.96×σ₀. In some examples, the self-consistency threshold value may be 0.7977. In some examples, the abnormality detection of the gold-standard consistency threshold value may be performed by obtaining target gold-standard consistency of annotation doctors with different threshold values and calculating a gold-standard consistency mean μ₁ and a gold-standard consistency variance σ₁, and under the assumption that the target gold-standard consistency satisfies a Gaussian distribution, the gold-standard consistency threshold value may be μ₁−1.96×σ₁. In some examples, the gold-standard consistency threshold value may be 0.6235. The detailed description may refer to the relevant description of step S160, which will not be repeated here.

In some examples, absolute majority voting is used to compare each annotation result of each target fundus image in the plurality of groups of target annotation results 140 to determine a final annotation result 150 for each target fundus image. Specifically, when absolute majority voting is used to compare individual annotation result, the annotation results will be accepted as part of the final annotation result 150 if more than half of the annotation results are consistent (i.e., more than half of the valid votes are required to be accepted). In some examples, if the final annotation result 150 cannot be determined (i.e., the number of valid votes is not more than half), the target fundus image is annotated as a difficult fundus image. In some examples, difficult fundus images may be annotated and arbitrated to obtain a final annotation result 150. Thus, the final annotation result 150 may be obtained based on the absolute majority voting method. The detailed description may refer to the relevant description of step S170, and will not be repeated here.

However, the examples of the present disclosure are not limited hereto, in other examples, gathering may be comparing respective annotation results in the plurality of groups of target annotation results 140 for respective target fundus images. In some examples, in the case that the respective annotation results are consistent, the annotation results may be taken as the final annotation result 150 for the target fundus image. In some examples, in the case that the plurality of annotation results are inconsistent, if the plurality of annotation results simultaneously include the same determination result and only one annotation result includes a determination result which is not identified in other annotation results, the target fundus image may be annotated as a fundus image to be quality controlled, otherwise, the target fundus image may be annotated as a difficult fundus image. In this case, by comparing the respective annotation results of the respective target fundus images in the plurality of groups of target annotation results 140, the target fundus image may be classified into a target fundus image having a final annotation result 150, a fundus image to be quality-controlled, and a difficult fundus image, and the final annotation result 150 may be obtained. In some examples, quality control may be performed on the fundus images to be quality controlled. In some examples, if it is determined that an unidentified determination result does not exist, the same determination result may be taken as the final annotation result 150. In some examples, if it is determined that an unidentified determination result exists, the fundus image to be quality-controlled may be annotated as a difficult fundus image. In some examples, difficult fundus images may be annotated and arbitrated to obtain a final annotation result 150. Thus, the final annotation result 150 of the difficult fundus image may be obtained. The detailed description may refer to the relevant description of step S170, and will not be repeated here.

As described above, in some embodiments, the quality control methods described herein are performed by computerized quality control system 300. In some embodiments, quality control system 300 is a single computer system, and in other embodiments quality control system 300 is a plurality of networked computer systems. In a networked arrangement, system 300 may operate as one or more servers with one or more client machine in a server-client network environment, or in a peer-to-peer/distributed network environment. The various computer systems may comprise one or more server computers, client computers, personal computers, tablets, or any other machine capable of executing software that performs the described quality control methods of this disclosure. In some embodiments, the system 300 comprises one or more programmable processors, one or more executable software programs stored within non-transitory computer-readable media, one or more user interfaces for inputting annotation results, and one or more means of transmitting collected data throughout the individual computerized elements of the system 300.

The quality control methods described herein can be executed by the one or more processors through sequential or parallel operations at the speed inherent to a computer processor, such that quality control operations can be performed on hundreds of thousands of fundus images in near real time (perceived as simultaneous or instant execution) from the perspective of a human user; allowing for collection, storage, and analysis of a vast collection of fundus images in a manner which far exceeds the capabilities of the human mind. As the methods and systems described herein are configured to perform quality control analysis for thousands of images and to store continuously improving gold-standard sets 220 and self-consistency determination data sets 230, the overall effectiveness of the quality control system improves over time via machine learning. The resulting effect of this learning is a continual increase to the accuracy of data annotations for fundus images. Other methods of further increasing processing power and optimizing the capabilities of the quality control system 300, including integration with other systems and forms of AI and machine learning, are contemplated and may be implemented in additional embodiments.

Although the present disclosure has been particularly shown and described with reference to the accompanying drawings and examples, it is to be understood that the disclosure is not limited in any manner by the foregoing description. Modifications and variations of the present disclosure, as required, may be made by those skilled in the art without departing from the true spirit and scope of the present disclosure and are intended to be within the scope of the disclosure.

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

1. A computerized quality control method for data annotation on a fundus image, comprising:
acquiring a plurality of fundus images, where the plurality of fundus images is up to 200,000 fundus images; and
performing a series of quality control steps, comprising:
performing standardization processing on each of the plurality of fundus images to obtain a plurality of standardized fundus images;
performing preliminary filtering on quality of each of the plurality of standardized fundus images to obtain a plurality of qualified fundus images;
preparing a target fundus image set, wherein the target fundus image set comprises a data set to be calibrated comprising the plurality of qualified fundus images, a gold-standard data set comprising a first preset number of gold-standard fundus images with a known correct annotation result, and a self-consistency determination data set composed of at least one image in the data set to be calibrated, and taking each image of the target fundus image set as a respective target fundus image;
annotating respective images of the target fundus image set by a plurality of first annotation doctors respectively to obtain a plurality of groups of doctor annotation results, wherein the doctor annotation results comprise at least one determination result and the determination result comprises at least disease information of no obvious abnormality or of a disease;
calculating self-consistency and gold-standard consistency of corresponding first annotation doctors based on the doctor annotation results to acquire the doctor annotation results of the first annotation doctors satisfying a preset condition as target annotation results, wherein:
the self-consistency is obtained by taking any one of two groups of annotation results of the doctor annotation result of each image in the self-consistency determination data set and the doctor annotation result of an image, which is repeated with respective image in the self-consistency determination data set, in the data set to be calibrated as a first group of annotation results and taking another group as a second group of annotation results and performing evaluation using a self-consistency determination and evaluation method, and
the gold-standard consistency is obtained by taking the correct annotation result of the gold-standard data set as a first group of annotation results and the doctor annotation result of each image in the gold-standard data set as a second group of annotation results and using a gold-standard consistency determination and evaluation method; and
gathering a plurality of sets of the target annotation results to obtain a final annotation result.

2. The computerized quality control method according to claim 1, wherein:
the preset condition is that the self-consistency is greater than a self-consistency threshold value; and
the gold-standard consistency is greater than a gold-standard consistency threshold value.

3. The computerized quality control method according to claim 2, wherein:
target self-consistency and target gold-standard consistency of doctors with different threshold value annotation are analyzed; and
abnormality detection comprises determining the self-consistency threshold value and the gold-standard consistency threshold value.

4. The computerized quality control method according to claim 3, wherein:
the abnormality detection comprises acquiring the target self-consistency of the doctors with different threshold value annotation and calculate a self-consistency mean value μ₀ and a self-consistency variance Go, under assumption that the target self-consistency satisfies a Gaussian distribution, the self-consistency threshold value is μ₀−1.96×σ₀; and
the abnormality detection comprises acquiring the target gold-standard consistency of the doctors with different threshold value annotation and calculate a gold-standard consistency mean value μ₁ and a gold-standard consistency variance σ1, under assumption that the target gold-standard consistency satisfies a Gaussian distribution, the gold-standard consistency threshold value is μ1−1.96×σ₁.

5. The computerized quality control method according to claim 1, wherein the doctor annotation result of the first annotation doctor which does not meet the preset condition is re-annotated by the second annotation doctor on each image in the target fundus image set until the doctor annotation result meeting the preset condition is obtained as the target annotation result.

6. The computerized quality control method according to claim 1, wherein:
the self-consistency determination method comprises calculating a disease self-consistency of each first annotation doctor determining each disease by a quadratic weighted kappa coefficient and weighs each disease self-consistency to calculate the self-consistency of each first annotation doctor; and
the gold-standard consistency determination method comprises calculating the gold-standard consistency of a disease of each first annotation doctor determining each disease using a quadratic weighted kappa coefficient and weighs the gold-standard consistency of the disease to calculate the gold-standard consistency of each first annotation doctor.

7. The computerized quality control method according to claim 6, wherein:
the quadratic weighted kappa coefficient κ is
$$κ = 1 - \frac{{\sum }_{i , j} {W}_{ij} {X}_{ij}}{{\sum }_{i , j} {W}_{ij} {E}_{ij}} ,$$
Wᵢⱼ represents a quadratic weighting coefficient, Xᵢⱼ represents a number of the target fundus images for which the determination result in the first group of annotation results is i and the determination result in the second group of annotation results is j, and Eᵢⱼ represents an expected number of the target fundus images for which the determination result in the first group of annotation results is i and the determination result in the second group of annotation results is j.

8. The computerized quality control method according to claim 1, wherein:
the gathering comprises comparing each annotation result of each the target fundus image in a plurality of groups of the target annotation results using an absolute majority voting method to determine the final annotation result of each the target fundus image, and if the final annotation result is not able to be determined, the target fundus image is annotated as a difficult fundus image; and
the difficult fundus image is annotated and arbitrated to obtain the final annotation result.

9. The computerized quality control method according to claim 1, wherein:
the gathering comprises comparing each annotation result of each target fundus image in the plurality of groups of the target annotation results;
if each annotation result is consistent, taking the annotation result as the final annotation result of the target fundus image, while if the plurality of annotation results are inconsistent;
if the plurality of annotation results simultaneously comprise a same determination result and only one annotation result comprises a determination result which is not identified in other annotation results, the target fundus image is annotated as a fundus image to be quality-controlled; and
otherwise, the target fundus image is annotated as a difficult fundus image; and
quality control is performed on the fundus image to be quality-controlled and the final annotation result is obtained, and the difficult fundus image is annotated and arbitrated to obtain the final annotation result.

10. The computerized quality control method according to claim 1, wherein:
in the preliminary filtering, the quality of the standardized fundus image is determined by a plurality of first annotation doctors to classify the standardized fundus image into a plurality of image quality grades; and
the qualified fundus image is the standardized fundus image of which the image quality grade is qualified.

11. The computerized quality control method according to claim 10, wherein:
the standardized fundus image are ranked based on factors that affect the quality of the fundus image; and
the factors affecting the quality of the fundus image comprise at least one of location at which the fundus image was taken, exposure, and definition.

12. The computerized quality control method according to claim 1, wherein:
in the annotation, each image of the target fundus image set is classified into three image quality grades of qualified, barely qualified, and unqualified; and
the qualified fundus image is the standardized fundus image with an image quality grade of qualified and barely qualified.

13. The computerized quality control method according to claim 1, wherein the disease comprises at least one of diabetic retinopathy, hypertensive retinopathy, glaucoma, retinal vein occlusion, retinal artery occlusion, age-related macular degeneration, high myopia macular degeneration, retinal detachment, optic nerve disease, and congenital abnormalities of disc development.

14. The computerized quality control method according to claim 1, wherein:
the preset condition is d_{self}≤D and d_{gold}≤D, wherein d_{self} is a self-evaluation index based on the self-consistency, d_{gold} is a gold-standard evaluation index based on the gold-standard consistency, and D is an evaluation index threshold value;
the self-evaluation index d_{self} satisfies following formula:
d_{self}=|J_{self}−κ_{self}|/κ_{self}×100%, wherein J_{self}=SE_{self}+SP_{self}−1, SE_{self} is, sensitivity of the first annotation doctor obtained based on the two groups of annotation results for evaluating the self-consistency, SP_{self} is specificity of the first annotation doctor obtained based on the two groups of annotation results for evaluating the self-consistency, and κ_{self} is the self-consistency of the first annotation doctor; and
the gold-standard evaluation index d_{gold} satisfies following formula:
d_{gold}=|J_{gold}−κ_{gold}|/K_{gold}×100% wherein J_{gold}=SE_{gold}+SP_{gold}−1 SE_{gold} is, sensitivity of the first annotation doctor obtained based on the two groups of annotation results for evaluating the gold-standard consistency, and SP_{gold} is specificity of the first annotation doctor obtained based on the two groups of annotation results for evaluating the gold-standard consistency, and κ_{gold} is the gold-standard consistency of the first annotation doctor.

15. A computerized quality control system for data annotation on a fundus image, comprising:
a plurality of modules having a plurality of functions, the modules comprising:
an acquisition module configured to acquire a plurality of fundus images, where the plurality of fundus images is up to 200,000 fundus images;
a standardization processing module configured to perform standardization processing on each of the plurality of fundus images to obtain a plurality of standardized fundus images;
a preliminary filtering module configured to perform preliminary filtering on quality of each of the standardized fundus images to obtain a plurality of qualified fundus images;
a data preparation module configured to prepare a target fundus image set, wherein the target fundus image set comprises a data set to be calibrated comprising the plurality of qualified fundus images, a gold-standard data set comprising a first preset number of gold-standard fundus images with a known correct annotation result, and a self-consistency determination data set composed of at least one image in the data set to be calibrated, each image of the target fundus image set is taken as each target fundus image;
an annotation module configured to acquire a plurality of groups of doctor annotation results by a plurality of first annotation doctors respectively annotating each image in the target fundus image set, wherein the doctor annotation results comprise at least one determination result, the determination result at least comprises disease information of no obvious abnormality or of a disease;
an evaluation module configured to calculate a self-consistency and a gold-standard consistency of a corresponding first annotation doctor based on the doctor annotation result to obtain the doctor annotation result of the first annotation doctor satisfying a preset condition as a target annotation result, wherein:
the self-consistency is obtained by taking any one of two groups of annotation results of the doctor annotation result of each image in the self-consistency determination data set and the doctor annotation result of an image, which is repeated with respective image in the self-consistency determination data set, in the data set to be calibrated as a first group of annotation results and another group as a second group of annotation results and performing evaluation using a self-consistency determination and evaluation method, and
the gold-standard consistency is obtained by taking the correct annotation result of the gold-standard data set as a first group of annotation results and the doctor annotation result of each image in the gold-standard data set as a second group of annotation results and using a gold-standard consistency determination and evaluation method; and
a gathering module configured to gather the plurality of groups of the target annotation results to obtain a final annotation result.

16. The computerized quality control system according to claim 15, wherein:
the preset condition is that the self-consistency is greater than a self-consistency threshold value; and
the gold-standard consistency is greater than a gold-standard consistency threshold value.

17. The computerized quality control system according to claim 16, wherein:
target self-consistency and target gold-standard consistency of doctors with different threshold value annotation are analyzed;
abnormality detection comprises determining the self-consistency threshold value and the gold-standard consistency threshold value;
the abnormality detection comprises acquiring the target self-consistency of the doctors with different threshold value annotation and calculate a self-consistency mean value po and a self-consistency variance Go, under assumption that the target self-consistency satisfies a Gaussian distribution, the self-consistency threshold value is μ₀−1.96×σ₀; and
the abnormality detection comprises acquiring the target gold-standard consistency of the doctors with different threshold value annotation and calculate a gold-standard consistency mean value μ₁ and a gold-standard consistency variance σ₁, under assumption that the target gold-standard consistency satisfies a Gaussian distribution, the gold-standard consistency threshold value is μ₁−1.96×σ₁.

18. The computerized quality control system according to claim 15, wherein:
the self-consistency determination and evaluation method comprises calculating a disease self-consistency of each first annotation doctor determining each disease by a quadratic weighted kappa coefficient and weighting each disease self-consistency to calculate the self-consistency of each first annotation doctor; and
the gold-standard consistency determination and evaluation method comprises calculating the gold-standard consistency of a disease of each first annotation doctor determining each disease using a quadratic weighted kappa coefficient and weighting the gold-standard consistency of the disease to calculate the gold-standard consistency of each first annotation doctor.

19. The computerized quality control system according to claim 18, wherein:
the quadratic weighted kappa coefficient κ is
$$κ = 1 - \frac{{\sum }_{i , j} {W}_{ij} {X}_{ij}}{{\sum }_{i , j} {W}_{ij} {E}_{ij}} ,$$
Wᵢⱼ represents a quadratic weighting coefficient, Xᵢⱼ represents a number of the target fundus images for which the determination result in the first group of annotation results is i and the determination result in the second group of annotation results is j, and Eᵢⱼ represents an expected number of the target fundus images for which the determination result in the first group of annotation results is i and the determination result in the second group of annotation results is j.

20. The computerized quality control system according to claim 15, wherein:
the preset condition is d_{self}≤D and d_{gold}≤D, wherein d_{self} is a self-evaluation index based on the self-consistency, d_{gold} is a gold-standard evaluation index based on the gold-standard consistency, and D is an evaluation index threshold value;
the self-evaluation index d_{self} satisfies following formula:
d_{self}=|J_{self}−κ_{self}|/κ_{self}×100%, wherein J_{self}=SE_{self}+SP_{self}−1, SE_{self} is, sensitivity of the first annotation doctor obtained based on the two groups of annotation results for evaluating the self-consistency, SP_{self} is specificity of the first annotation doctor obtained based on the two groups of annotation results for evaluating the self-consistency, and κ_{self} is the self-consistency of the first annotation doctor; and
the gold-standard evaluation index d_{gold} satisfies following formula:
d_{gold}=|J_{gold}−κ_{gold}|/κ_{gold}×100%, wherein J_{gold}=SE_{gold}+SP_{gold}−1, SE_{gold}, is sensitivity of the first annotation doctor obtained based on the two groups of annotation results for evaluating the gold-standard consistency, and SP_{gold} is specificity of the first annotation doctor obtained based on the two groups of annotation results for evaluating the gold-standard consistency, and κ_{gold} is the gold-standard consistency of the first annotation doctor.
